(19) United States
(12) Patent Application Publication
(10) Pub. No.: US 2021/0000734 A1
(43) Pub. Date: Jan. 7, 2021

(54) A COMPOSITION SUITABLE FOR HAIR CARE

(71) Applicant: BASF SE, Ludwigshafen (DE)

(72) Inventors: Anna Maria Mueller-Cristadoro, Lemfoerde (DE); Olivier Fleischel, Ludwigshafen (DE); Catherine Weichold, Düsseldorf-Holthausen (DE); Christina Kohlmann, Monheim (DE); Bjoern Klotz, Düsseldorf-Holthausen (DE); Sybille Cornelsen, Monheim (DE)

(21) Appl. No.: 16/978,215

(22) PCT Filed: Mar. 8, 2019
(86) PCT No.: PCT/EP2019/055811

§ 371 Date: Sep. 4, 2020

(30) Foreign Application Priority Data
Mar. 15, 2018 (EP) 18162092.3

(51) Int. Cl. A61K 8/88; A61K 8/20; A61Q 5/00; A61Q 5/02; A61Q 5/12

(52) U.S. Cl. CPC A61K 8/88; A61K 8/20; A61Q 5/002; A61K 2800/596; A61Q 5/12; A61K 2800/544; A61Q 5/02

(57) ABSTRACT

The present invention relates to a composition suitable for treating hair comprising a polymer selected from the group consisting of a branched polylysine and a branched polylysine in which to 80% of the amino groups present in the branched polylysine are derivatized so that they bear one or two substituents, and comprising at least one surfactant. Furthermore, the present invention relates to a branched polylysine in which 1 to 80% of the amino groups present in the branched polylysine are derivatized so that they bear one or two substituents, wherein these substituents are acyl groups having 6 to 24 C-atoms. Furthermore, the present invention relates to the use of a branched polylysine or of the branched polylysine in which amino groups are derivatized or of the said composition for treating hair. Furthermore, the present invention relates to a process for treating hair comprising contacting the hair with a branched polylysine or with the branched polylysine in which amino groups are derivatized or with the said composition.

The present invention relates to a composition suitable for treating hair comprising a polymer selected from the group consisting of a branched polylysine and a branched polylysine in which 1 to 80% of the amino groups present in the branched polylysine are derivatized so that they bear one or two substituents, and comprising at least one surfactant. Furthermore, the present invention relates to a branched polylysine in which 1 to 80% of the amino groups present in the branched polylysine are derivatized so that they bear one or two substituents, wherein these substituents are acyl groups having 6 to 24 C-atoms. Furthermore, the present invention relates to the use of a branched polylysine or of the branched polylysine in which amino groups are derivatized or of the said composition for treating hair. Furthermore, the present invention relates to a process for treating hair comprising contacting the hair with a branched polylysine or with the branched polylysine in which amino groups are derivatized or with the said composition.

The surface of hair, especially of human hair, can be damaged by hair treatment, e. g. by bleaching. Therefore, there is a need for substances that have a repair effect on damaged hair.

An indication of hair repair is the denaturation temperature of human hair (Tₘₐₓ). The denaturation temperature of human hair protein can be determined as described by Wortmann et al. (J. Appl. Polym. Sci. volume 48 (1993), page 137) using differential scanning calorimetry with a heating rate of 2K/min. Denaturation temperatures are expressed as Tₘₐₓ, i.e. the temperature of the denaturation peak maximum.

Untreated, virgin hair has a higher Tₘₐₓ than a damaged hair (e.g. bleached hair). Hereinafter, a “hair repair effect” can be determined by and thus can be defined as an increase of the Tₘₐₓ of (damaged) hair.

Lysine is an amino acid. There are two enantiomers of lysine, L-lysine and D-lysine. Lysine has two amino groups, one at the alpha-position, one at the epsilon-position. Linear L-polylysine is produced by natural fermentation and it is referred to as epsilon-Polylysine or ε-Polylysine. This fermentation can be carried out with bacteria strains of the genus Streptomyces, e. g. Streptomyces albulus.

Branched polylysine is formed when both amino-groups of lysine (alpha and epsilon) react in a polycondensation reaction with the carboxylic acid group of lysine.

WO 2007/060119, US 201/0123148, and WO 2016/062578 disclose a process for making branched polylysine. Derivatives of branched polylysine are also disclosed. Inter alia, polylysine reacted with saturated or unsaturated long-chain carboxylic acids is disclosed (WO 2016/062578, page 9, lines 29 to 30).

WO 2013/013889 discloses the use of hyperbranched polylysine as shale inhibitor.

The use of linear (not branched) polylysine in hair care applications is known.

EP 2 036 536 A1 discloses the use of polyamines in conditioning applications (hair care (curly hair) and antifrizz application). polylysine is mentioned.

EP 1 563 826 A1 describes the use of polylysine in spray applications (having a conditioner effect).

JP 62221616 describes the use of polylysine in hair cosmetic applications having a conditioning effect. The material obtained by bacteria is linear and in epsilon form.

JP 2007169192 and JP 2007153791 describe hair cosmetics and shampoo formulations respectively with polylysine or its salt in combination with quaternized amines, again for conditioning applications.

WO 2015/128566 describes a treatment which consists in the application of polyamines (polylysine is mentioned) to keratin fibers and thereafter the application of an activated ester to the creatine fibers. Repair effects are mentioned.

US 20090074700 describes the use of polyamines (polylysine is mentioned) together with acids and insoluble material to impart shine on hair.

Parameters to characterize branched polylysine (apart from the differentiation between L- and D-lysine) are the average molar mass, either the number average Mn or the weight average Mw, the polydispersity PD=Mw/Mn (measured via gel permeation chromatography), the degree of branching, and the amine number. These parameters can be determined by methods known in the art and/or as disclosed in WO 2016/062578. In general, it is assumed that the backbone of branched polylysine as well as the side chains of branched polylysine both have amide groups formed by alpha-amino groups and by epsilon amino-groups. The relative amount of alpha- and epsilon amide groups in the backbone and in the side chains is not known.

The problem underlying the present invention is to provide a substance and/or a composition having a hair repair effect, i. e. providing a substance and/or a composition that is capable of increasing the denaturation temperature of human hair (Tₘₐₓ), especially if this denaturation temperature has been decreased before by damaging the hair.

A first solution to this problem, and therefore a first subject of the present invention is a composition suitable for treating hair comprising a polymer selected from the group consisting of a branched polylysine and a branched polylysine in which 1 to 80% of the amino groups present in the branched polylysine are derivatized so that they bear one or two substituents, wherein these substituents are selected from the group consisting of
acyl groups having 6 to 24 C-atoms,
substituents having the formula —CH₂—CH(OH)—R or the formula —CHR—CH₂(OH), wherein
R is an alkyl moiety with 2 to 20 C-atoms,
substituents having the formula —C(═O)—NH—R, wherein R is an alky moiety with 2 to 20 C-atoms,
substituents of the formula —CH₂—CH₂—C(═O)OR¹, wherein R¹ is an alkyl group having 6 to 24 C-atoms,
substituents formed by the reaction of the amino groups present in the branched polylysine with polyisobutylene succinic anhydride (PIBSA),
and mixtures thereof,

and comprising at least one surfactant selected from the group consisting of an anionic surfactant, a cationic surfactant, a nonionic surfactant, a zwitterionic surfactant, and mixtures thereof, and optionally comprising one or more further cosmetically acceptable ingredients different from the branched polylysine, different from the branched polylysine in which amino groups are derivatized, and different from the surfactant.

Branched polylysine according to the present invention is a polylysine that comprises at least one repeating unit per polylysine-molecule which is a lysine-moiety and which is bound to another lysine-moiety via its alpha-amino group and to yet another lysine-moiety via its epsilon-amino group.

A further solution to this problem, and therefore a further subject of the present invention is a branched polylysine in which 1 to 80%, preferably 1 to 30%, more preferably 1 to 20%, and especially 4 to 15%, of the amino groups present in the branched polylysine are derivatized so that they bear one or two substituents, wherein these substituents are acyl groups having 6 to 24 C-atoms, preferably acyl groups having 10 to 20 C-atoms, more preferably 18 C-atoms.

A further subject of the present invention is the use of a branched polylysine or of the branched polylysine in which amino groups are derivatized according to the present invention, or of the composition according to the present invention for treating hair (preferably human hair).

A further subject of the present invention is a process for treating hair, preferably human hair, comprising contacting the hair with a branched polylysine or with a branched polylysine in which amino groups are derivatized according to the present invention, or with the composition according to the present invention.

Preferred embodiments of the subjects of the present invention are given in the dependent claims of the present text.

“A composition suitable for treating hair” according to the present invention can be any composition suitable for treating hair. It can be a composition suitable for cleansing hair, especially a shampoo, it can be a composition for conditioning hair (a conditioner), it can be a mask.

The meaning of hair conditioning is known to the person skilled in the art. It is described in US 2017/0333734 (BASF internal reference PF 77681 US02) in paragraph [0007].

In the branched polylysine according to the present invention and in the branched polylysine according to the present invention in which amino groups are derivatized the lysine repeating units can be derived from L-lysine or from D-lysine or from a mixture of L- and D-lysine, especially from a racemic mixture. Preferably these repeating units are derived from L-lysine.

The branched polylysine according to the present invention and the branched polylysine according to the present invention in which amino groups are derivatized (in the latter case based on/calculated with respect to the polylysine framework, without derivatization) typically have the following properties:
The number average of the molar mass Mn is 300 to 5000 g/mol.
The weight average of the molar mass MW is 500 to 10000 g/mol, preferably 1000 to 5000 g/mol.
The polydispersity PD (=Mw/Mn) is 1.2 to 4, preferably 1.2 to 3, more preferably 1.2 to 2.5
The amine number is 350 to 600 mgKOH/g, preferably 350 to 500 mgKOH/g.
The degree of branching DB is 0.15 to 0.5, preferably 0.2 to 0.35, more preferably 0.2 to 0.3.

The definition of amine number and of “degree of branching” DB can be found in the examples section of the present text.

The branched polylysine according to the present invention can be made by heating a mixture comprising lysine and water, especially as described in WO 2016/062578.

The branched polylysine in which 1 to 80%, preferably 1 to 30%, more preferably 1 to 20%, and especially 4 to 10%, of the amino groups present in the branched polylysine are derivatized so that they bear one or two substituents, as it is defined in the claims of the present text, can be made by reacting branched polylysine with carboxylic acids, esters of carboxylic acids, anhydrides of carboxylic acids, epoxy compounds, isocyanates, or with compounds obtainable via Michael addition of amino groups to Michael donor fatty chains (e. g. acrylates or methacrylates), or with PIBSA (polyisobutylene succinic anhydride).

Typically, the branched polylysine in which 1 to 80%, preferably 1 to 30%, more preferably 1 to 20%, and especially 4 to 10%, of the amino groups present in the branched polylysine are derivatized so that they bear one or two substituents, as it is defined in the claims of the present text, have the following properties:
The number average of the molar mass Mn is 300 to 5000 g/mol.
The weight average of the molar mass MW is 500 to 10000 g/mol, preferably 1000 to 5000 g/mol.
The polydispersity PD (=Mw/Mn) is 1.2 to 4, preferably 1.2 to 3, more preferably 1.2 to 2.5
The amine number is 350 to 600 mgKOH/g, preferably 350 to 500 mgKOH/g.
The degree of branching DB is 0.15 to 0.5, preferably 0.2 to 0.35, more preferably 0.2 to 0.3.

The definition of amine number and of “degree of branching” DB can be found in the examples section of the present text.

According to the present invention the surfactant can be any surfactant. It can be selected from the group consisting of an anionic surfactant, a cationic surfactant, a nonionic surfactant, a zwitterionic surfactant, and mixtures thereof. The surfactants according to the present invention can be selected amongst the surfactants described in US 2017/0333734 (BASF internal reference PF 77681 US02) in paragraphs [0029] to [0032].

Further cosmetically acceptable ingredients different from the branched polylysine, different from the branched polylysine in which amino groups are derivatized, and different from the surfactant, can be any cosmetically acceptable ingredients known to the person skilled in the art. These further cosmetically acceptable ingredients can be selected amongst the ingredients described in US 2017/0333734 (BASF internal reference PF 77681 US02) in paragraphs [0033] to [0066].

### EXAMPLES

### Methods and Definitions

Concentrations

% means % by weight, unless defined differently.

Degree of Branching (DB)

The degree of branching (DB) of branched polylysine is defined according to H. Frey et al., Acta Polymer., 48, pages 30 to 35 (1997) as

DB[%]=100×2D/(2D+L)

wherein D denotes the fraction of dendritic units and L denotes the fraction of linear units in the sample that is concerned.

DB was determined by ¹H NMR.

Amine Number

The amine number (unit: mg KOH/g), also referred to as amino number, was determined by titration. It was determined as described in WO 2016/062578 according to the formula given on page 13 of WO 2016/062578:
Calculation:

$$Amine number = \frac{( {V}_{P} - {V}_{B} ) \cdot t \cdot c \cdot 56.1}{E} = \frac{( {V}_{P} - {V}_{B} ) \cdot t}{E} \cdot 5.61$$

with
Amine number=Fraction of total amine, calculated as mg KOH/g
V_{P}=Consumption of standard solution up to the inflection point [nil]
V_{B}=Consumption of standard solution in blank value titration [ml]
t=titer of the standard solution
c=concentration of the standard solution [=, 0.1 mol/I]
56.1=molar weight of KOH [g/mol]
E=weight of sample taken [g].

Average Molar Mass Mn (Number Average) and Mw (Weight Average)

Mn and Mw were determined by gel permeation chromatography as described in WO 2016/062578 (see page 12 of WO 2016/062578, wherein trifluoroacete means trifluoroacetic acid):

M_{w} and Mₙ were determined by size exclusion chromatography under the following con-ditions:

Solvent: 0. (w/w) trifluoroacetate, 0.1 M NaCl in distilled water

Flow: 0.8 ml/min

Injection volume: 100 μl

Column material: hydroxylated polymethacrylate (TSKgel G3000PWXL)

Calibration: poly(2-vinylpyridine) standards in the molar mass range from 839 to 1.020.000 g/mole (from PSS, Mainz, Germany)

Polydispersity

PD is defined as PD=Mw/Mn

### Experiments Carried Out

### Example 1: Branched Polylysine 1

500 g of a 50% aqueous solution of L-Lysine were placed in a 21 four-necked flask equipped with a stirrer, a condensation column, a thermometer and a nitrogen inlet. The L-Lysine solution was heated to the boiling point. Then the temperature of the external heat source was increased according to the following profile: 1 h at 150° C., 1 h at 160, 1 h at 170 and 1 h at 180° C. while water was distilled off. The pressure was then decreased to 200 mbar while the external heat source was maintained at 180° C. The reaction for circa 2 hours continued under vacuum (200 mbar). The reaction melt was cooled at 120° C. and dissolved in 234 g of water. The polymer was characterized by gel permeation chromatography, determination of viscosity, pH, solid content, degree of branching and amino number:

Mn: 2150 g/mol

Mw: 4110 g/mol

PD: 1.9

viscosity (25° C.): 200 mPas (Rheomat, dynamic viscosity 100*1/sec)

pH: 10-11

solid content: 50.5%

degree of branching measured by ¹H-NMR=0.25

amino number: 185 mg KOH/g for the solution (390 mg KOH/g of polylysine neat)

### Example 2: Branched Polylysine 2

500 g of a 50% aqueous solution of L-Lysine were placed in a 21 four-necked flask equipped with a stirrer, a condensation column, a thermometer and a nitrogen inlet. The L-Lysine solution was heated to the boiling point. Then the temperature of the external heat source was increased according to the following profile: 1 h at 150° C., 1 h at 160, 1 h at 170 and 1 h at 180° C. while water was distilled off. The pressure was then decreased to 200 mbar while the external heat source was maintained at 180° C. The reaction for circa 1 hour continued under vacuum (200 mbar). The reaction melt was cooled at 120° C. and dissolved in 242 g of water. The polymer was characterized by gel permeation chromatography, determination of viscosity, pH, solid content, degree of branching and amino number:

Mn: 1350 g/mol

Mw: 2590 g/mol

PD: 1.9

viscosity. (25° C.): 110 mPas

pH: 11-12

solid content: 49.7%

degree of branching measured via ¹H-NMR=0.24

amino number: 215 mg KOH/g (433 mg KOH/g polylysine neat)

### Example 3: Branched Polylysine 3

1700 g of a 50% aqueous solution of L-Lysine were placed in a 21 four-necked flask equipped with a stirrer a condensation column, a thermometer and a nitrogen inlet. The L-Lysine solution was heated to the boiling point. Then the temperature of the external heat source was increased according to the following profile: 1 h at 150° C., 1 h at 160, 1 h at 170 and 1 h at 180° C. while water was distilled off. The pressure was then decreased to 200 mbar while the external heat source was maintained at 180° C. and the reaction for circa 1+½ hours continued under vacuum (200 mbar). The warm reaction melt was collected in an aluminum vessel. At room temperature, a solid material was obtained.

The polymer was characterized by gel permeation chromatography, degree of branching and amino number:

Mn: 2150 g/mol

Mw: 3650 g/mol

PD: 1.7

degree of branching measured via ¹H-NMR=0.28

amino number: 410 mg KOH/g

### Example 4: Branched Polylysine 4

1700 g of a 50% aqueous solution of L-Lysine were placed in a 21 four-necked flask equipped with a stirrer a condensation column, a thermometer and a nitrogen inlet. The L-Lysine solution was heated to the boiling point. Then the temperature of the external heat source was increased according to the following profile: 1 h at 150° C., 1 h at 160, 1 h at 170 and 1 h at 180° C. while water was distilled off. The pressure was then decreased to 200 mbar while the external heat source was maintained at 180° C. and the reaction for circa 2 hours continued under vacuum (200 mbar). The warm reaction melt was collected in an aluminum vessel. At room temperature, a solid material was obtained.

The polymer was characterized by gel permeation chromatography, degree of branching and amino number:

Mn: 2070 g/mol

Mw: 4070 g/mol

PD: 2

degree of branching measured via ¹H-NMR=0.3

amino number: 366 mgKOH/g

### Example 5: Branched Polylysine Modified with Oleic Acid (Modification of 4.8% of the Amino End Groups with Oleic Acid)

250 g of branched polylysine made according to the procedure described in example 2 (not dissolved in water) was melted at 120° C. and 26.37 g oleic acid were added under stirring. The pressure was then decreased to 200 mbar while the external heat source was maintained at 180° C. and the reaction continued for circa 1 hour under vacuum (200 mbar). At room temperature, a solid material was obtained.

The polymer was characterized by gel permeation chromatography:

Mn: 1710 g/mol

Mw: 2930 g/mol

PD: 1.7

### Example 6: Branched Polylysine Modified with Oleic Acid (Modification of 10% of the Amino End Groups with Oleic Acid)

50 g of branched polylysine, made according to the procedure described in example 3 (not dissolved in water) was melted at 120° C. and 10.25 g oleic acid were added under stirring. The pressure was then decreased to 200 mbar while the external heat source was maintained at 180° C. and the reaction continued for circa 1 hour under vacuum (200 mbar). At room temperature, a solid material was obtained.

### Application Examples

The denaturation temperatures of human hair proteins were determined as described by Wortmann et al. (J. Appl. Polym. Sci. 48 (1993) 137) using a heating rate of 2K/min. Denaturation temperatures are expressed as Tₘₐₓ, i.e. the temperature of the denaturation peak maximum. The higher this temperature is the better. In case of damaged hair this denaturation temperature is lower than in case of virgin, not damaged, hair. If the denaturation temperature of damaged hair can be increased by treating it with a hair care composition this indicates a hair repair effect. Table 1 summarizes the DSC-data obtained.

TABLE 1
DSC data on virgin hair, 3 times bleached hair, and hair that has
been bleached 3 times and after this treated with an aqueous polymer
solution having a polymer-concentration of 1% by weight.

| Appl. | | Tₘₐₓ | |
|---|---|---|---|
| Example | Sample | [° C.] | Comments |
| 1 | virgin hair | 141.8 | |
| 2 | placebo | 133.2 | 3 times bleached then treated with |
| | | | water |
| 3 | with | 140.9 | 3 times bleached then treated with |
| | example 1 | | the polylysine of example 1 |
| 4 | With | 140.4 | 3 times bleached then treated with |
| | example 2 | | the polylysine of example 2 |
| 5 | with | 141.8 | 3 times bleached then treated with |
| | example 3 | | the polylysine of example 3 |
| 6 | with | 142.2 | 3 times bleached then treated with |
| | example 4 | | the polylysine of example 4 |
| 7 | with | 142.6 | 3 times bleached then treated with |
| | example 5 | | the polylysine of example 5 which |
| | | | is branched polylysine modified |
| | | | with oleic acid |
| 8 | comparative | 138.9 | 3 times bleached then treated with |
| | example 1 | | linear epsilon-polylysine (from |
| | | | Zhengzhou Bainafo Bioengineering |
| | | | Co. Ltd.) |

The results show that treatment with branched polylysine results in a repair effect. This repair effect is higher than the repair effect achieved with linear epsilon-polylysine (comparative example 1). The repair effect of branched polylysine modified with oleic acid (according to example 5) is the best repair effect achieved amongst all samples tested.

### Examples of Formulations

The following formulations have been made. Their pH and their viscosity was determined.

### Formulation 1: A Sulfate-Free Soft Shampoo

| | INCI | % | % active matter surfactants | |
|---|---|---|---|---|
| Plantapon ® SF | Sodium Cocoamphoacetate, | 40.00 | Sodium Cocoamphoacetate | 4-8% |
| | Glycerin, | | Lauryl Glucoside | 2-6% |
| | Lauryl Glucoside, | | Sodium Cocoyl Glutamate | <2% |
| | Sodium Cocoyl Glutamate, | | Sodium Lauryl Glucose | <2% |
| | Sodium Lauryl Glucose | | Carboxylate | |
| | Carboxylate | | | |
| Arlypon ® TT | PEG/PPG-120/10 | 0.50 | | |
| | Trimethylolpropane | | | |
| | Trioleate, Laureth-2 | | | |
| Cosmedia ® | Polyquaternium-37 | 0.10 | | |
| Ultragel 300 | | | | |
| Branched polylysine | Polylysine | 1.00 | | |
| from example 4 | | | | |
| Lamesoft ® PO 65 | Coco-Glucoside, | 1.00 | Coco-Glucoside | 0.2-0.4% |
| | Glyceryl Oleate | | | |
| Water, demin. | Aqua | ad 100 | | |
| Citric Acid | Citric Acid | qs | | |

pH value 5.6 (measured with a pH-meter Schott Instrument Lab 850 at 22° C.)
Viscosity 9028 mPas (rpm 60 s64-22° C.) (measured with a digital Brookfield viscometer, 60 rotations per minute, spindle no. s64, measuring temperature was 22° C.)

### Formulation 2: A Mild Baby Wash

| | INCI | % | % active matter surfactants | |
|---|---|---|---|---|
| Plantapon ® PSC | Coco-Glucoside, | 20.00 | Coco-Glucoside | 6-10% |
| | Disodium Lauryl | | Disodium Lauryl | 1-3% |
| | Sulfosuccinate, | | Sulfosuccinate | |
| | Glycerin | | | |
| Plantacare ® | Cocamidopropyl | 20.00 | Cocamidopropyl | 8.8-9.2% |
| 2000 UP | Betaine | | Betaine | |
| | Decyl Glucoside | 2.00 | Decyl Glucoside | 1.0-1.2% |
| Lamesoft ® PO 65 | Coco-Glucoside, | 1.00 | Coco-Glucoside | 0.2-0.4% |
| | Glyceryl Oleate | | | |
| Gluadin ® WLM | Laurdimonium | 1.00 | | |
| | Hydroxypropyl | | | |
| | Hydrolyzed Wheat | | | |
| | Protein | | | |
| Branched polylysine | Polylysine | 1.00 | | |
| from example 4 | | | | |
| Water, demin. | Aqua | ad 100 | | |
| Citric Acid | Citric Acid | qs | | |

pH value 5.6 (measured with a pH-meter Schott Instrument Lab 850 at 22° C.)
Viscosity 4909 mPas (rpm 60 s64-22° C.) (measured with a digital Brookfield viscometer, 60 rotations per minute, spindle no. s64, measuring temperature was 22° C.)

### Formulation 3: An Argan Oil Hair Conditioner

| | INCI | % | % active matter emulsifiers | |
|---|---|---|---|---|
| Dehyquart ® A-CA | Cetrimonium Chloride | 1.00 | Cetrimonium Chloride | 0.2-0.3% |
| Emulgade ® 1000 NI | Cetearyl Alcohol, | 4.00 | Ceteareth-20 | 0.4-0.8% |
| | Ceteareth-20 | | | |
| Lipofructyl ™ | Argania Spinosa | 2.00 | | |
| Argan LS 9779 | Kernel Oil | | | |
| Branched polylysine | Polylysine | 1.00 | | |
| from example 4 | | | | |
| Glycerin | Glycerin | 2.00 | | |
| Water, demin. | Aqua | ad 100 | | |

pH value 3.8 (measured with a pH-meter Schott Instrument Lab 850 at 22° C.)
Viscosity <100 mPas (measured with a digital Brookfield viscometer, 60 rotations per minute, spindle no. s64, measuring temperature was 22° C.)

1. A composition suitable for treating hair
comprising a polymer selected from the group consisting of
a branched polylysine
and a branched polylysine in which 1 to 80% of the amino groups present in the branched polylysine are derivatized so that they bear one or two substituents, wherein these substituents are selected from the group consisting of
acyl groups having 6 to 24 C-atoms,
substituents having the formula —CH₂—CH(OH)—R or the formula —CHR—CH₂(OH), wherein R is an alkyl moiety with 2 to 20 C-atoms,
substituents having the formula —C(═O)—NH—R, wherein R is an alkyl moiety with 2 to 20 C-atoms,
substituents of the formula —CH₂—CH₂—C(═O)OR¹, wherein R¹ is an alkyl group having 6 to 24 C-atoms,
substituents formed by the reaction of the amino groups present in the branched polylysine with polyisobutylene succinic anhydride (PIBSA),
and mixtures thereof,
and comprising at least one surfactant selected from the group consisting of an anionic surfactant, a cationic surfactant, a nonionic surfactant, a zwitterionic surfactant, and mixtures thereof.
and optionally comprising one or more further cosmetically acceptable ingredient different from the branched polylysine, different from the branched polylysine in which amino groups are derivatized, and different from the surfactant.

2. The composition according to claim 1, wherein the polymer is a branched polylysine in which amino groups are derivatized, wherein the substituents are selected from the group consisting of
acyl groups having 10 to 20 C-atoms,
substituents having the formula —CH₂—CH(OH)—R or the formula —CHR—CH₂(OH), wherein R is an alkyl moiety with 2 to 20 C-atoms,
substituents having the formula —C(═O)—NH—R, wherein R is an alkyl moiety with 2 to 20 C-atoms,
substituents of the formula —CH₂—CH₂—C(═O)OR¹, wherein R¹ is an alkyl group having 6 to 24 C-atoms,
substituents formed by the reaction of the amino groups present in the branched polylysine with polyisobuthylene succinic anhydride (PIBSA),
and mixtures thereof.

3. The composition according to claim 1, wherein the composition is suitable for cleansing hair or suitable for conditioning hair.

4. The composition according to claim 1 comprising
0.1 to 10% by weight, of the polymer,
0.1 to 30% by weight, of the surfactant, and
0 to 99.8% by weight, of the one or more further cosmetically acceptable ingredient.

5. The composition according to claim 1 comprising
0.5 to 1.5% by weight by weight of the polymer,
6 to 19% by weight by weight of the surfactant, and
0 to 93.5% by weight by weight of the one or more further cosmetically acceptable ingredient,
or comprising
0.5 to 1.5% by weight by weight of the polymer,
16 to 24% by weight by weight of the surfactant, and
0 to 83.5% by weight by weight of the one or more further cosmetically acceptable ingredient,
or comprising
0.5 to 1.5% by weight by weight of the polymer,
0.5 to 1.2% by weight by weight of the surfactant, and
0 to 99% by weight by weight of the one or more further cosmetically acceptable ingredient.

6. The composition according to claim 1 comprising not more than 5% by weight inorganic salt.

7. The composition according to claim 1 comprising 5% by weight sodium chloride.

8. A branched polylysine in which 1 to 80% of the amino groups present in the branched polylysine are derivatized so that they bear one or two substituents,
wherein these substituents are acyl groups having 6 to 24 C-atoms.

9. The branched polylysine in which amino groups are derivatized according to claim 8 wherein the substituents are acyl groups derived from oleic acid.

10. (canceled)

11. The process according to claim 12 wherein the hair to be treated is damaged and wherein the treating brings about a hair repair effect.

12. A process for treating hair, comprising contacting the hair with a branched polylysine or with a branched polylysine in which amino groups are derivatized, as defined claim 8.

13. A process for treating hair, comprising contacting the hair with a composition of claim 1.

14. The branched polylysine of claim 8 in which 1 to 20% of the amino groups are derivatized.

15. The branched polylysine of claim 8 in which 4 to 20% of the amino groups are derivatized.

16. The branched polylysine of claim 8 in which the acryl group has 10 to 20 C-atoms.
